(19) United States
(12) Patent Application Publication
(10) Pub. No.: US 2006/0130838 A1
(43) Pub. Date: Jun. 22, 2006

(54) Data logger for monitoring asthmatic conditions

(76) Inventors: Yong Yeow Lee, Singapore (SG); Hugo Van Bever, Singapore (SG); Victor D. Samper, Singapore (SG)

Correspondence Address: BARNES & THORNBURG, 11 SOUTH MERIDIAN, INDIANAPOLIS, IN, 46204, US

(21) Appl. No.: 11/017,154

(22) Filed: Dec. 20, 2004

(51) Int. Cl. A61M 11/00; A62B 9/00

(52) U.S. Cl. 128/205.23; 128/200.23

(57) ABSTRACT

An apparatus for monitoring asthmatic conditions takes the form of a data logger for attaching to a spacer. The spacer has a chamber with an input end and an output end and defining an interior space wherein the input end receives a medication discharge from a discharge orifice of a canister of medication into the interior space and wherein the medication can be withdrawn from the interior space by inhalation by a patient from the output end. The data logger is mounted to the spacer and may include such sensors as pressure transducers and accelerometers. The sensors may be arranged to measure various aspects of the environment of the spacer, such as a pressure change induced by the inhalation of the medicine from the chamber. A memory may also be included for recording the measurements for later transfer to another device for processing and analysis.

### FIELD OF THE INVENTION

The present invention relates to administration of treatment for asthmatic conditions and, more particularly, to apparatus for monitoring asthmatic conditions and compliance to a prescribed treatment regimen.

### BACKGROUND

There are several challenges encountered in the diagnosis and monitoring of asthmatic conditions among young children and infants.

Diagnosis of asthma is typically improved through an interview with a patient. Unfortunately, when the patient is a young child or an infant, the patient is usually unable to correctly relate the extent of the symptoms of asthma to a medical practitioner. As such, the judgment of the practitioner may be adversely affected.

Even when a child is old enough to self administer asthma treating medicines; the child may forget to take the medicines. Such inconsistent administration may render inconclusive efforts to determine whether the medicines prescribed have been beneficial.

During a visit with a medical practitioner, breathing metrics, e.g., tidal volume and breathing rate, may be measured to assist in assessing the extent to which a patient is affected by asthma. However, the benchmarks for such metrics are known to vary from one child to another. Indeed, the measurements are only meaningful relatively to previous measurements of the same metrics in the same patient.

Adrenergic bronchodilators are medicines that are breathed in through the mouth to open up the bronchial tubes (air passages) of the lungs. Some of these medicines are used to treat the symptoms of asthma, chronic bronchitis, emphysema, and other lung diseases, while others are used to prevent the symptoms of such diseases. Adrenergic bronchodilators are often administered from a metered-dose inhaler.

Notably, a spacer is often used with an inhaler for administering medicines employed in the treatment of asthma for young children and infants. It has been identified that young children, and even parents, often do not use the spacer correctly during medicine administration. When the spacer is used improperly, the amount of asthma treating medicine administered to a patient may be less than prescribed.

Clearly, it would be helpful, both for general practitioners and for specialists, to have a reliable manner in which to determine a given patient's compliance to a prescribed treatment regimen. Additionally, it would be helpful to provide feedback to a user of a spacer to indicate proper operation.

### SUMMARY

An apparatus is attached to a spacer for use with an inhaler for administering medicines employed in the treatment of asthma. Through recording environmental measurements related to the administration of the medicine, the apparatus may be arranged to monitor compliance of the patient to a prescribed treatment regimen. The apparatus may also be arranged to record a breathing pattern of the patient during each use. From the recorded breathing pattern, a degree of asthma may later be determined. Additionally, an accelerometer may be attached to the spacer to monitor and indicate, to the user of the spacer, proper operation.

In accordance with an aspect of the present invention there is provided an apparatus for monitoring asthmatic conditions, wherein the apparatus is arranged for mounting to a spacer, the spacer having an input end, an output end and a mask mounted at the output end, and wherein an application of the mask to a face of a patient creates a cavity. The apparatus includes an enclosure, a conduit for pneumatically coupling the enclosure to the cavity such that pressure changes in the cavity result in pressure changes in the enclosure and a pressure transducer mounted in fluid communication with the cavity for: sensing the pressure changes in the cavity and producing an electrical output signal representative of the pressure changes. The apparatus also includes an analog to digital converter for converting the electrical output signal representative of the pressure changes to a digital value representative of the pressure changes, a memory and a processor communicatively connected to the analog to digital converter and the memory for: receiving the digital value representative of the pressure changes; and transmitting, to the memory, the digital value representative of the pressure changes.

In accordance with another aspect of the present invention there is provided an apparatus for monitoring asthmatic conditions, wherein the apparatus is arranged for mounting to a spacer. The apparatus includes a frame and an accelerometer mounted to the frame for: sensing a change in pitch of the apparatus; and producing an electrical output signal representative of the change in pitch. The apparatus also includes an analog to digital converter for converting the electrical output signal representative of the change in pitch to a digital value representative of the change in pitch, a memory and a processor communicatively connected to the analog to digital converter and the memory for: receiving the digital value representative of the change in pitch; and transmitting, to the memory, the digital value representative of the change in pitch.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate example embodiments of this invention:

FIG. 1 illustrates an exploded perspective view of a data logger for monitoring asthmatic conditions according to an embodiment of the present invention, along with a spacer and a metered-dose inhaler;

FIG. 2 schematically illustrates the components of the data logger of FIG. 1; and

FIG. 3 illustrates a computer station, capable of running companion software to analyze data transferred to the computer station from the data logger of FIG. 1 according to methods exemplary of the present invention.

### DETAILED DESCRIPTION

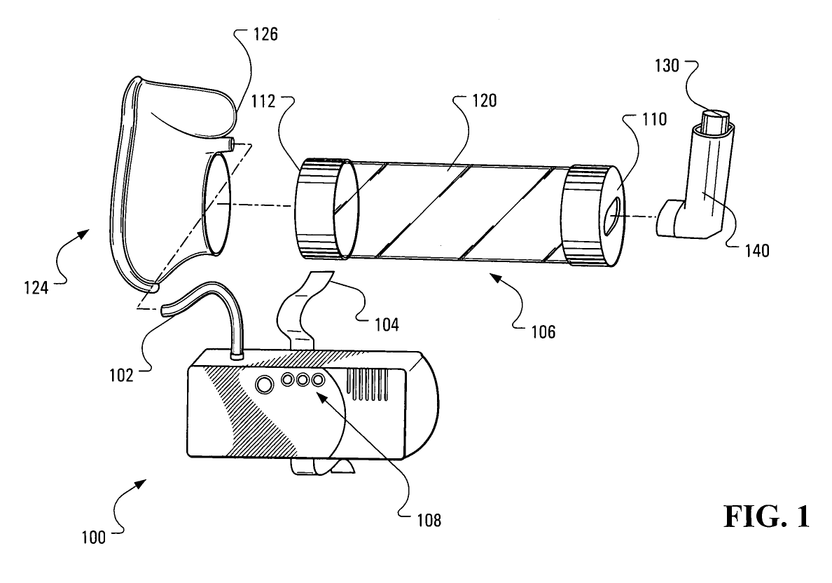

FIG. 1 illustrates a data logger 100 for monitoring asthmatic conditions attached, by way of a strap 104, to the side of a spacer 106. The spacer 106, as is typical of such known devices for facilitating the administration of an asthma treating medicine contained in a canister 130 mounted within a metered-dose inhaler 140, includes a chamber 120 with an input end 110 and an output end 112 defining an interior space. The spacer 106 also includes a mask 124 at the output end 112 of the chamber 120 for fitting over the mouth and nose of a patient. Between the output end 112 of the chamber 120 and the mask 124, an inhalation valve (not shown) allows the passage of air and asthma treating medicine toward the patient, yet resists allowing the passage of breath exhaled from the patient. Instead, the breath exhaled from the patient may be allowed to pass through an exhalation valve 126 located in the mask 124. Components of the data logger 100 are housed within an enclosure or frame. The mask 124 may be provided with an aperture for allowing some of the breath exhaled from the patient to pass through a conduit 102 to an interior of the enclosure of the data logger 100. FIG. 1 also illustrates a set of LEDs 108 mounted in the data logger 100.

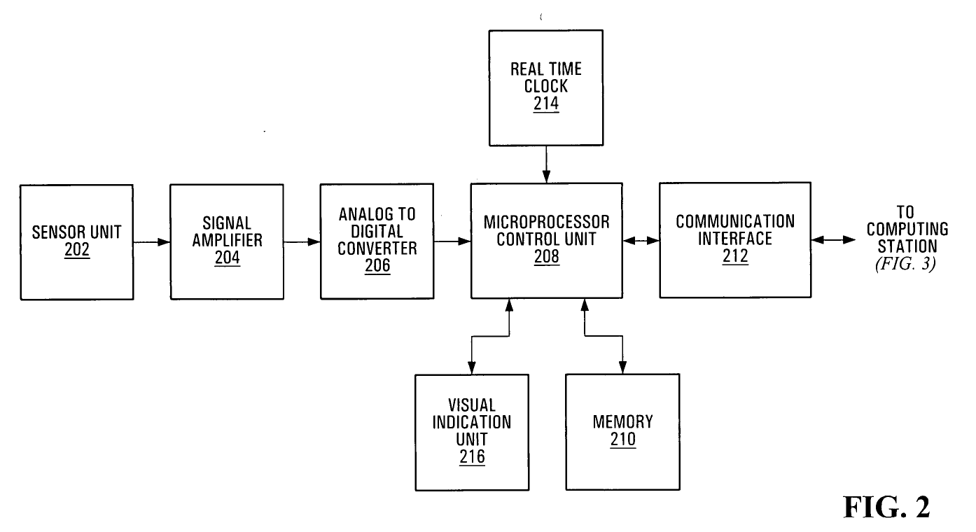

The data logger 100 is illustrated schematically in FIG. 2 to include a sensor unit 202 whose output is passed to a signal amplifier 204. The output of the amplifier 204 is passed to an analog-to-digital converter (ADC) 206 whose digital output is received by a microprocessor control unit (MCU) 208. The MCU 208 maintains a connection to a long-term memory 210, a communication interface 212, a real time clock 214 and a visual indication unit 216. Although not shown, the MCU 208 may include a short-term volatile random access memory.

The long-term memory 210 is preferably a removable, non-volatile form of memory, such as the standard Multi-Media Card or Secure Digital Card. The communication interface 212 may be, for instance, a standard RS232 (EIA232) or Universal Serial Bus (USB) interface. In one embodiment of the invention, the sensor unit 202 includes a pressure transducer, such as the known Motorola Integrated Pressure Sensor MPXV5004DP. In another embodiment of the invention, the sensor unit 202 includes an accelerometer. In a further embodiment of the invention, the sensor unit 202 includes both a pressure transducer and an accelerometer. The illustrated visual indication unit 216 may, for example, be associated with the LEDs 108 of FIG. 1 or, alternatively, may be associated with a liquid crystal display or other manner of relaying status information to a user.

As would be expected, the data logger 100 includes a power source (not shown), such as a rechargeable lithium-ion battery, to provide power to the various elements of the data logger 100. In addition, the data logger 100 may include a power management system (not shown) for monitoring and controlling the discharging and recharging of the power source.

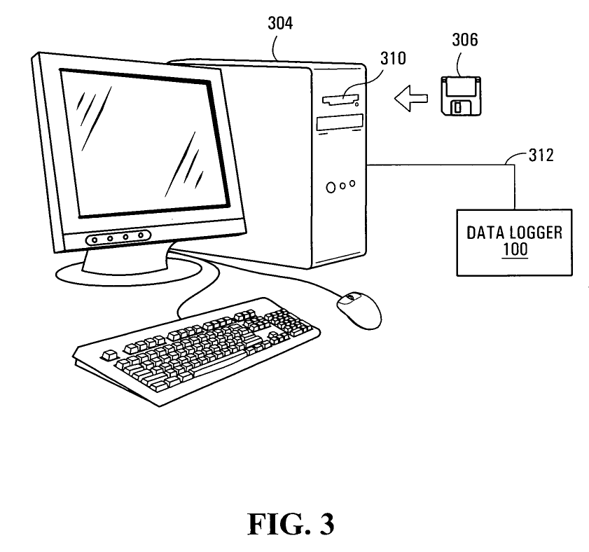

A computing station 300, capable of analyzing data received from the data logger 100 according to methods exemplary of the present invention, is illustrated in FIG. 3. The computing station 300 includes a display monitor 302 and a central processing unit 304. The central processing unit 304 may include hardware to network with other computers, long term and short term memory and a processor. As is typical, connected to the central processing unit 304 may be input peripherals including a keyboard 308. The computing station 300 may be loaded with companion software for executing methods exemplary of this invention from a software medium 306 which could be a disk, a tape, a chip or a random access memory containing a file downloaded from a remote source.

In one embodiment of an aspect of the present invention, the computing station receives data from the data logger 100 over an interface cable 312 connected to the communication interface 212 of the data logger 100. In another embodiment of an aspect of the present invention, the long-term memory 210 is removable non-volatile memory such as a memory card. The long-term memory 210 may be removed from the data logger 100 and placed into a memory card reader 310 for reading by the computing station 300. The computing station 300 receives data from the data logger 100 by using the memory card reader 310 to read the data from the memory card. In yet another embodiment of an aspect of the present invention, both the computing station 300 and the communication interface 212 are configured for wireless communication using an infrared interface or the known Bluetooth™ technology.

During a typical administration of asthma treating medicine, a patient using the spacer 106 with the metered-dose inhaler 140 activates the metered-dose inhaler 140 to expel asthma treating medicine from the canister 130 into the interior space of the chamber 120. The patient then breathes deeply inward (inspiration cycle) to inhale the asthma treating medicine from the interior space of the chamber 120 and subsequently exhales (expiration cycle) into the mask 124.

In operation, before administration of asthma treating medicine, the user may activate the data logger 100 by pressing a power button. It may be easily shown that during the inspiration and expiration cycles of the patient, pressure changes occur in a cavity between the face of the patient and the mask 124 of the spacer 106. These pressure changes in the cavity may be experienced within the enclosure of the data logger 100 through the pneumatic coupling of the cavity to the enclosure of the data logger 100 via the conduit 102. Alternatively, the cavity may be pneumatically coupled to an interior of the sensor unit 202 within the enclosure. Changes in the environment of the data logger 100 may be sensed by sensors (not shown) within the sensor unit 202. For example, periodic pressure measurements may be taken at a pressure transducer in the sensor unit 202. An analog signal representative of each pressure measurement made at the sensor unit 202 may be amplified at the amplifier 204 and converted, at the ADC 206, to a digital value representative of the pressure measurement. The digital value received from the ADC 206 by the MCU 208 may then be recorded, by the MCU 208, initially in the short-term memory and subsequently in the long-term memory 210. The digital value representative of the pressure measurement may be associated with a date and time provided by the real time clock 216 that is associated with the MCU 208.

At a later time, the digital values representative of the pressure measurements that are recorded on the long-term memory 210 may be transmitted via the communication interface 212 and the interface cable 312 to the computing station 300 (FIG. 3). The computing station 300 may be associated with the patient or with a medical practitioner. The computing station 300 may include a memory for receiving the digital values representative of the pressure measurements and a processor for processing the digital values to convert the digital values into flow measurements for use in quantitatively determining breathing rates and tidal volume.

In another embodiment of aspects of the present invention, movement of the data logger 100, and, by extension, the spacer 106 and the canister 130, may be measured by the sensor unit 202 by way of an accelerometer. While the accelerometer may be capable of measuring changes in yaw, pitch and roll of the data logger 100, the important measurement is the pitch of the data logger 100. That is, the spacer 106, and, therefore, the data logger 100, should be held horizontal during administration of the medicine. An analog signal representative of the pitch may be amplified at the amplifier 204 and converted, at the ADC 206, to a digital value representative of the pitch measurement. The digital values received from the ADC 206 by the MCU 208 may then be recorded, by the MCU 208, initially in the short-term memory and subsequently in the long-term memory 210.

The recorded pitch measurements may eventually be transmitted from the long-term memory 210 to the computing station via the communication interface 212, where the recorded pitch measurements may be analyzed to determine whether the chamber 120 has been held sufficiently horizontal during medicine administration. Additionally, movement measurements from the accelerometer may be analyzed at the before medicine administration.

The pitch measurements may be processed by the MCU 208 as they are received and a result of such processing may be used to drive the set of LEDs 108 to provide an indication the user of whether the spacer 106 is being held level.

The digital value representative of the movement measurement may be associated with a date and time provided by the real time clock 216 that is associated with the MCU 208.

In a simple use of the data logger 100, each activation of the data logger 100 may cause the time and date of the associated administration of the medicine to be recorded so that a practitioner, or a parent, of a patient may monitor compliance of the patient to a prescribed treatment regimen.

The digital values representative of the movement measurements may be processed on board the data logger 100 by the MCU 208 such that the result of such processing may be used to drive the set of LEDs 108 on the data logger 100 to provide feedback to the patient in the form of visual cues that indicate proper and improper movement of the spacer 106 during medicine administration.

The set of LEDs 108 on the data logger 100 may provide visual cues to the patient throughout the administration of medicine. For example, consider a three LED display with a yellow “not ready” LED, a red “error” LED and a green “finished” LED. When preparing to administer medicine, the user will insert the discharge end of the metered-dose inhaler 140 into the input end 110 of the chamber 120 and power on the data logger 100. When first powered on, the not ready LED may be turned on for a short period, say two seconds, while the data logger performs a hardware functionality check and logs an initial set of digital values from the sensor unit 202. The initial set of digital values may be used to develop an average, or baseline, sensor measurement against which subsequent measurements may be compared. Subsequent to the initialization of the data logger 100, the not ready LED may be turned off.

It is considered that the user will then shake the metered-dose inhaler 140 and, by extension, shake the spacer 106 and the data logger 100. The set of LEDs 108 may remain off for a predetermined waiting period to allow for the shaking. The predetermined waiting period may have duration of, for instance, five seconds.

Subsequent to the expiration of the predetermined waiting period, the MCU 208 of the data logger 100 may begin recording, in the short-term memory, the digital values, received from the ADC 206, that are representative of measurements made at the sensor unit 202. While the digital value recording is taking place, the not ready LED may be turned on and off in a blinking pattern. Where the measurements made at the sensor unit 202 are pressure change measurements, the finished LED may be arranged to blink on and off under control of the MCU 208 responsive to the pressure change measurements exceeding a predetermined threshold. Such blinking of the finished LED may be seen by the patient as being responsive to each breath. The digital value recording may continue for a predetermined logging period. The predetermined logging period may have duration of, for instance, 60 seconds.

Subsequent to the expiration of the predetermined logging period, the MCU 208 of the data logger 100 may record, in the long-term memory 210, the digital values that have been stored in the short-term memory. Upon completion of the recording of the digital values in the long-term memory 210, the finished LED may be turned on for a predetermined completion indication period. The predetermined completion indication period may have duration of, for instance, five seconds.

The set of LEDs 108 may also indicate various error conditions to the user. For instance, where the long-term memory 210 is removable, all three LEDs 108 may blink for three seconds when the data logger 100 is powered on and the long-term memory 210 is not present. Similarly, the error LED and the finished LED may blink for three seconds upon power on to indicate a low battery. Additionally, the error LED and the finished LED may be made to blink for ten seconds upon power on to indicate that the hardware functionality check has failed.

Where the long-term memory 210 is removable and is removed during recording of digital values by the MCU 208, the error LED may be arranged to blink until the long-term memory 210 is re-inserted or until a ten-second re-insertion period expires, whichever event occurs first. If the re-insertion period expires, the data logger 100 may be reset and all three LEDs 108 may be turned on briefly (say, for three seconds) to indicate the reset to the user.

If, during the first, say, ten seconds of the predetermined logging period, the digital values received at the MCU 208 do not differ significantly from the baseline measurement, the MCU 208 may decide that the data logger was unintentionally turned on and may, as a consequence, stop logging digital values to the short-term memory and may not record the values from the short-term memory to the long-term memory 210. Additionally, the MCU 208 may turn on all three LEDs 108 briefly to indicate cancellation of logging.

A user may also cancel unintended logging by pressing and holding the power button for, say, two seconds. Responsive to this action by the user, the MCU 208 may stop logging digital values to the short-term memory and may not record the values from the short-term memory to the long-term memory 210. Additionally, the MCU 208 may turn on all three LEDs 108 briefly to indicate cancellation of logging.

If the long-term memory 210 is a removable memory card, the card may include a unique identity of the patient readable by the data logger 100. In addition, each card may not have the same outward appearance. For instance, each card may be adorned with a photograph of the patient with which the card is associated or be labeled with patient identification information. The patient may have more than one card, for when the data for a measurement period exceeds the capacity of a single card. A secondary card may include an indication of the secondary nature in the patient identification information or may be adorned with a photograph of the patient with which the card is associated and a parent (i.e., second card maps to two people).

The data logger 100 may be arranged to expect a card for a specific patient. Where a card is inserted into the data logger that does not identify the patient as expected by the data logger 100, all three LEDs 108 may be arranged to blink on and off for a duration of three seconds.

At the end of the predetermined logging period, and before recording, in the long-term memory 210, the digital values that have been stored in the short-term memory, the MCU 208 may determine the memory required to record the digital values and the memory available in the long-term memory 210. If the memory required exceeds the memory available in the long-term memory 210, the MCU 208 may cause the LEDs 108 to blink for, say, a ten-second period, to indicate insufficient available memory. The blinking of the LEDs 108 may be discontinued before the end of the ten-second period if, for instance, the long-term memory 210 is removable and removed ahead of replacement with a new memory card.

The digital values representative of measurements made at the sensor unit 202 may be recorded to the long-term memory 210 each time the medicine in the canister 130 is administered over a measurement period. In a preferred embodiment, the measurement period is set at three months. However, as will be clear to a person of ordinary skill in the art, the measurement period may configured to have a longer or shorter duration, dependent upon the goals of those that are configuring the data logger 100 and the capacity of the long-term memory 210.

The long-term memory 210 may be configured to maintain information regarding the patient such as the identity of the patient and information regarding the prescription in the present canister 130 and previous canisters. The information regarding the prescription in previous canisters may also be limited by the goals of those that are configuring the data logger 100 and the capacity of the long-term memory 210. As discussed previously, the long-term memory 210 may be in the form of a memory card that may be removed from the data logger 100 and provided to the medical practitioner or specialist at the time of a patient visit. The medical practitioner may insert the memory card into the memory card reader 310 associated with the computing station 300 to update a patient profile.

There may be developed two versions of software to accompany the data logger 100, one version for the parents of young patients and the other version for general practitioners and specialists.

The version for general practitioners and specialists may execute on a central server (not shown) that is linked to the same network as the computing station 300. At each visit, the practitioner may upload measurements from the long-term memory 210 to a central server (not shown). A compliance chart may be generated to display, for a given month, a chronological indication of the pressure measurements and/or the movement measurements.

The version for general practitioners and specialists may be arranged to plot average breathing rate and tidal volume in charts so that a correlation can be drawn between the prescription and any benefit to the patient. Where the digital values representative of the measurements are recorded in a portable, secure storage medium, information sharing is possible, allowing multiple doctors to discuss and provide diagnosis and treatment collectively.

The version for patient caregivers may allow the digital values representative of the measurements that are received from the data logger 100 at the computing station 300 to be sent via electronic mail to computer account associated with a medical practitioner. This remote transmission of digital values representative of the measurements to a practitioner may be seen not only to simplify the process of diagnosis, but also to ensure the quality of each diagnosis.

Where the data logger 100 is provided separately from the spacer 106, the mask 124 of the spacer 106 may require adaptation to allow the data logger 100 to record pressure changes. In particular, a pin hole aperture may be placed in the mask 124 to allow the conduit 102 to pass through the mask 124 and thereby facilitate a transmission of pressure changes within the cavity between the mask 124 and the face of the patient to the data logger 100.

While FIG. 1 illustrates the data logger 100 attached to the side of a spacer 106, by way of the strap 104, it will be understood that the data logger 100 may be attached either permanently or temporarily to the spacer 106 in many other ways such as those that employ glue, tape, Velcro™ or magnets.

Other modifications will be apparent to those skilled in the art and, therefore, the invention is defined in the claims.

We claim:

1. An apparatus for monitoring asthmatic conditions, wherein said apparatus is arranged for mounting to a spacer, said spacer having an input end, an output end and a mask mounted at said output end, and wherein an application of said mask to a face of a patient creates a cavity, said apparatus comprising:
an enclosure;
a conduit for pneumatically coupling said enclosure to said cavity such that pressure changes in said cavity result in pressure changes in said enclosure;
a pressure transducer mounted in fluid communication with said cavity for:
sensing said pressure changes in said cavity; and
producing an electrical output signal representative of said pressure changes;
an analog to digital converter for converting said electrical output signal representative of said pressure changes to a digital value representative of said pressure changes;
a memory; and
a processor communicatively connected to said analog to digital converter and said memory for:
receiving said digital value representative of said pressure changes; and
transmitting, to said memory, said digital value representative of said pressure changes.

2. The apparatus of claim 1 further comprising a real time clock in communication with said processor.

3. The apparatus of claim 1 further comprising a visual cue indicator communicatively connected to said processor so that said processor may activate said visual cue indicator to indicate information regarding operation of said apparatus.

4. The apparatus of claim 3 wherein said visual cue indicator is a light emitting diode.

5. The apparatus of claim 1 further comprising:
an accelerometer mounted to said apparatus for:
sensing a change in pitch of said apparatus; and
producing an electrical output signal representative of said change in pitch;
wherein said analog to digital converter is further for converting said electrical output signal representative of said change in pitch to a digital value representative of said change in pitch; and
wherein said processor is further for:
receiving said digital value representative of said change in pitch; and
transmitting, to said memory, said digital value representative of said change in pitch.

6. The apparatus of claim 1 wherein said memory is a volatile random access memory.

7. The apparatus of claim 6 further comprising a non-volatile memory.

8. The apparatus of claim 7 wherein said non-volatile memory may be removed from said apparatus.

9. The apparatus of claim 1 wherein said memory is a non-volatile memory.

10. The apparatus of claim 1 further comprising a means for attaching said apparatus to said spacer.

11. The apparatus of claim 10 wherein said means for attaching is a strap.

12. An apparatus for monitoring asthmatic conditions, wherein said apparatus is arranged for mounting to a spacer, said apparatus comprising:
a frame;
an accelerometer mounted to said frame for:
sensing a change in pitch of said apparatus; and
producing an electrical output signal representative of said change in pitch;
an analog to digital converter for converting said electrical output signal representative of said change in pitch to a digital value representative of said change in pitch;
a memory; and
a processor communicatively connected to said analog to digital converter and said memory for:
receiving said digital value representative of said change in pitch; and
transmitting, to said memory, said digital value representative of said change in pitch.
